(19) United States
(12) Patent Application Publication
(10) Pub. No.: US 2020/0261341 A1
(43) Pub. Date: Aug. 20, 2020

(54) NUTRITIVE COSMETIC AND HAIR STIMULATING COMPOUND

(71) Applicant: JESSICA VILLENA CAPELLE DO VALLE, Niteroi (BR)

(72) Inventor: JESSICA VILLENA CAPELLE DO VALLE, Niteroi (BR)

(21) Appl. No.: 16/427,327

(22) Filed: May 30, 2019

(30) Foreign Application Priority Data
Feb. 18, 2019 (BR) PI 1020190032529

(51) Int. Cl. A61K 8/49; A61K 8/97; A61Q 7/00

(52) U.S. Cl. CPC A61K 8/4953; A61Q 7/00; A61K 8/97

(57) ABSTRACT

The present invention discloses a hair care and nourishing cosmetic composite, which relates to a cosmetic product for the aid in the treatment of hair loss, baldness, alopecia, weakness of the threads among others, which is characterized by combining the auxina tricógena with caffeine, differentiating the product for female and male users, being formulas for each gender as follows:
Formula for the female audience: 70% of Auxina Tricógena 15%, 30% of Caffeine 3%,
Formula for Male use: 70% caffeine 3%, 30% Tricogen Auxin, in any of the compositions, a quantity of ml of Hydroalcoholic Solution is applied according to the need of each person.

### BACKGROUND OF THE INVENTION

### Field of the Invention

The present invention relates to technical field of cosmetics industry.

### CROSS-REFERENCE TO RELATED APPLICATIONS

This patent application is being filed under 35 U.S.C. § 111 and claims the benefit of the Brazilian Patent Application BR 10 2019 003252 9, filed Feb. 18, 2019, the contents of which are incorporated herein by reference.

### SUMMARY OF THE INVENTION

The present invention relates to “Hair Care and Nourishing Cosmetic Compound”, hereinafter referred to as the “cosmetic compound”, which, in its own name, refers to an effective cosmetic product for treatment of hair loss, baldness, alopecia, weakness of the threads among others. The cosmetic compound also acts satisfactorily for hair follicle, meeting needs and expectations of the clients in the effective way. In this way, cosmetic compound uses tricogen auxin with caffeine, in different proportions and determined according to the sex of the person. A formula for each gender is as follows:
Formula for female use: 70% of auxina tricogena 15%, 30% of Caffeine 3%,
Formula for male use: 70% caffeine 3%, 30% tricogen auxin, in any of the compositions, a quantity of ml of hydroalcoholic solution is applied according to the need of each person.

The purpose of this cosmetic compound is to perform the stimulation of the follicles allowing the increase of capillary growth and resurgence of the wires in the follicles that were inactive.

As is well known among experts in the field, nowadays there are numerous people who suffer with scalp problems, among the most important being hair loss, scalp failure, as well as correcting the problem of gifted people with little hair.

The main hair problems that this cosmetic composition seeks to solve are the following: little hair; problems with the scalp and hair loss.

### Description of Related Art

From the analysis of the problems found and aiming the placing on the market of a cosmetic compound, which meets the conditions of nutrition and stimulation of the scalp, previous searches were carried out next to the INPI Data Bank and in it were found the following prior art references:
BR 10 2015 032386-7, filed Dec. 23, 2015 and published Jun. 27, 2017 under the heading “COSMETIC COMPOSITION FOR HAIR TREATMENT”, describes a cosmetic composition for capillary treatment, which comprises a synergistic mixture between natural extracts, peptides salt and esters: (a) Vitex agnus; (b) Pisum sativum; (c) Ginkgo biloba; (d) Vitis vinifera; (e) Acetyl tyrosine; (f) Hexapeptide-1; (g) Glycyrrhizate Dipotassium; (h) PEG-6 Isoesterate/Hesperetine Laurato; in a cosmetically acceptable carrier, particularly used in the treatment of gray wires by increasing the expression of genes and proteins related to pigmentation.
P10621064-3, filed Dec. 7, 2006 and published 29 Nov. 2011 under the heading “COMPOSITION FOR CAPILLARY TREATMENT”, the present invention provides a composition for capillary treatment comprising a combination of sugar, an amino acid and a salt of an organic acid. The composition is particularly suitable for the treatment of hair that is dry, damaged and/or prone to problems with maneuverability.

From the comparative analysis between the documents found and the applicant's cosmetic composite, it can be seen that the product now described and claim in this application refers to a totally different cosmetic composition, which is why we consider that there are no technical or legal impediments for the granting a patent for the applicant's invention.

### DESCRIPTION OF THE INVENTION

The technological advance of this cosmetic composite aims to give an immediate answer to meet the patients' wishes, both genders, to avoid the problems previously described, the formula of this cosmetic composition combines auxina tricogena with caffeine, differentiating the product for female audience and male, the formulas being as follows:
Formula for the female audience:

70% Auxine Tricogen 15%

30% Caffeine 3%
Formula for Male Use:

70% caffeine 3%

Any of the above compositions is applied as hydroalcoholic solution with the amount of ml used according to the need of each person.

A main purpose of the cosmetic compound is to stimulate and activate the follicles by means of micro needle application of the reported composition, emphasizing that the results obtained are already obtained in the first session, showing fuller and brighter yarns and after the second it is already possible to verify the appearance of new hair from the follicles that no longer produced them.

It is noteworthy that the results happen regardless of the cause, and that the person must be with their blood and hormonal rates within normal and free of any disorder to have the result offered by applying the product.

It is worth mentioning that caffeine, when applied to the scalp, can act in various ways, which leads to greater nutrition of the yarns and, consequently, contributes to growth, with the following advantages being the following:
Antioxidant action;
Microcirculation induction;
Reduction of fat accumulation;
Inhibits the enzyme responsible for the production of the hormone that refines the wires;
Stimulates hair regeneration and
Helps in the revitalization of the hair follicle.

On the other hand, Auxina Tricogena can act in several ways when used in the scalp because it is a complex phytonic action that increases the redox processes, normalizing the metabolic and respiratory changes of the hair root, presenting the following advantages:
Stimulates the vascularization of the scalp allowing oxygenation of the area;
Increases the production of acidic mucopolysaccharides, chondroitin sulfate, hepatic hyaluronic acid in the anagen phase;
It does not interfere with the total physiological cycle of the scalp, in many products called capillary stimulants (such as rube facients, vasodilators and hormones).

1. Nutritive cosmetic and hair stimulating compound for aid in treatment of hair loss, baldness, alopecia, wire weakness, in stimulation of the follicles allowing the increase of capillary growth and resurgence of the wires in the follicles that were inactive, and others, obtained by combining tricogen auxin with caffeine, differentiating the product for female and male users, the formulas for each gender being the following:
formula for female users: 70% of Auxina Tricogena 15%, 30% of Caffeine 3%,
formula for male users: 70% caffeine 3%, 30% Tricogen Auxin, in any of the compositions, a quantity of ml of hydroalcoholic solution is applied according to the need of each person.
